(19) United States
(12) Patent Application Publication
(10) Pub. No.: US 2018/0325495 A1
(43) Pub. Date: Nov. 15, 2018

(54) MEDICAL IMAGE DIAGNOSTIC APPARATUS

(71) Applicant: Konica Minolta, Inc., Tokyo (JP)

(72) Inventors: Yasuhiro NAKAMURA, Sagamihara-shi (JP); Yosuke KIMURA, Tokyo (JP); Akiko SUGIYAMA, Tokyo (JP)

(21) Appl. No.: 15/959,768

(22) Filed: Apr. 23, 2018

(30) Foreign Application Priority Data
May 9, 2017 (JP) 2017-092724

(51) Int. Cl. A61B 8/08; G16H 30/20; A61B 8/00

(52) U.S. Cl. CPC A61B 8/5223; A61B 8/56; A61B 8/54; G16H 30/20

(57) ABSTRACT

A medical image diagnostic apparatus which processes a medical image of a subject is shown. The apparatus includes the following. A power supply supplies power to each unit of the medical image diagnostic apparatus. A power supply button receives operation to end or start the medical image diagnostic apparatus. A power supply controller stands by to receive input of the power supply button and supplies power to the power supply and controls the power supply. A battery is mounted in the medical image diagnostic apparatus and supplies power to the power supply controller. A switch receives operation to switch between on and off, and electrically connects or cuts a path between the battery and the power supply controller in response to the operation.

### BACKGROUND

### 1. Technological Field

The present invention relates to a medical image diagnostic apparatus.

### 2. Description of the Related Art

Conventionally, an ultrasound diagnostic apparatus which includes a battery and a AC-DC (Alternating Current-Direct Current) converter is well-known as a medical image diagnostic apparatus (refer to Japanese Patent Application Laid-Open Publication No. H5-261096). The AC-DC converter converts the source power supplied from external sources such as commercial power supply and supplies the power to the power supply which supplies power to each unit of the ultrasound diagnostic apparatus. Further, the AC-DC converter also charges the battery. When the supply from the external power supply cannot be received, the battery supplies power to the power supply of the apparatus. In normal diagnosis situations, the ultrasound diagnostic apparatus operates with the commercial power supply. However, if diagnosis is performed outside, for example, the commercial power supply cannot be used. In this case, the apparatus operates on batteries. After operating on batteries, the apparatus is connected to the commercial power supply and charged.

In recent ultrasound diagnostic apparatuses, even if the apparatus ends the process, a portion of the control circuits is provided with source power so as to prepare for the next time the apparatus is started. Here, a conventional ultrasound diagnostic apparatus 1D provided with a battery is described shortly with reference to FIG. 14. FIG. 14 is a block diagram showing a functional configuration of a conventional ultrasound diagnostic apparatus 1D in a standby state.

In FIG. 14, the double arrow shows a power supply line, the broken arrow shows a standby power supply line, and the single arrow shows a signal line or a control line. In FIG. 14, a bold line shows a power feed line or operating component, and a thin line shows a no-power feed line or non-operating component. Such notation is similar in the other diagrams.

As shown in FIG. 14, the ultrasound diagnostic apparatus 1D includes a housing 2D, an ultrasound probe 40, a display 50, an operating unit 60, and an AC adapter 70. The housing 2D includes a system controller 11, a power supply controller 13A, a power supply 14, a power supply button 15, a DC input connector 16, a battery 17, and an ultrasound signal processor 30. The system controller 11 includes a volatile memory 12. The ultrasound probe 40 is connected to the ultrasound signal processor 30 through a cable 41. The ultrasound signal processor 30 includes a transmitter/receiver 31, a beam former 32, a signal processor 33, and a display processor 34.

When the ultrasound diagnostic apparatus 1D is started, the power supply controller 13A supplies to the power supply 14 the source power supplied by the battery 17 or the commercial power supply through the AC adapter 70 connected to the commercial power supply. The power supply 14 supplies the source power to the system controller 11, the power supply button 15, the ultrasound signal processor 30, the ultrasound probe 40, the display 50, and the operating unit 60. The system controller 11 controls the ultrasound signal processor 30 to generate the ultrasound image data based on the ultrasound transmitted to the subject by the ultrasound probe 40 and received by the ultrasound probe 40. The ultrasound image data is displayed on the display 50.

There are two ways to end the ultrasound diagnostic apparatus 1D, standby and shutdown. FIG. 14 shows the ultrasound diagnostic apparatus 1D in a standby state in which the AC adapter 70 is not connected (no power supply from the commercial power supply). In the standby state in which the AC adapter 70 is not connected, in order to be able to use the data stored in the memory 12 after starting, the source power output from the battery 17 is supplied to the power supply controller 13A, the power supply 14, the memory 12 and the power supply button 15 as the standby power. The power supply controller 13A stands by for the pressing of the power supply button 15 to start the ultrasound diagnostic apparatus 1D.

In the shutdown state in which the AC adapter 70 is not connected, the data stored in the memory 12 is not used after starting. Therefore, the source power output from the battery 17 is supplied to the power supply controller 13A and the power supply button 15 as the standby power.

The ultrasound diagnostic apparatus 1D is able to access to the commercial power supply without difficulty in normal situations. When diagnosis is performed during disasters or in undeveloped areas, a few hours to a few days may pass after the battery 17 is charged with the commercial power supply, the apparatus is transported, and the diagnosis is started. In a situation where the battery cannot be charged not only during standby but also during shutdown, the power of the battery 17 is consumed gradually as standby power, and the amount of time that the apparatus can be used for diagnosis is shortened. As a simple solution, the charged battery 17 can be removed from the ultrasound diagnostic apparatus 1D to prevent discharge for standby power. However, the battery 17 needs to be moved separately, and the transporting of the apparatus becomes troublesome.

### SUMMARY

The present invention is conceived in view of the above problems, and an object of the present invention is to easily reduce the amount of consumed power after ending the apparatus.

To achieve at least one of the abovementioned objects, according to an aspect of the present invention, a medical image diagnostic apparatus reflecting one aspect of the present invention is described, the apparatus including, a power supply which supplies power to each unit of the medical image diagnostic apparatus; a power supply button which receives operation to end or start the medical image diagnostic apparatus; a power supply controller which stands by to receive input of the power supply button and which supplies power to the power supply and controls the power supply; a battery which is mounted in the medical image diagnostic apparatus and which supplies power to the power supply controller; and a switch which receives operation to switch between on and off, and which electrically connects or cuts a path between the battery and the power supply controller in response to the operation.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a block diagram showing a functional configuration of a first ultrasound diagnostic apparatus in a normal shutdown state according to a first embodiment of the present invention.

FIG. 2 is a diagram showing a schematic configuration of a first switch.

FIG. 3 is a block diagram showing a functional configuration of the first ultrasound diagnostic apparatus in a complete shutdown state.

FIG. 4 is a block diagram showing a functional configuration of the first ultrasound diagnostic apparatus in a charging state during normal shutdown.

FIG. 5 is a block diagram showing a functional configuration of a second ultrasound diagnostic apparatus in a normal shutdown state.

FIG. 6 is a diagram showing a schematic configuration of a relay.

FIG. 7 is a block diagram showing a functional configuration of the second ultrasound diagnostic apparatus in a complete shutdown state.

FIG. 8 is a block diagram showing a functional configuration of the second ultrasound diagnostic apparatus in a charging state during normal shutdown.

FIG. 9 is a block diagram showing a functional configuration of the second ultrasound diagnostic apparatus in a standby state.

FIG. 10 is a block diagram showing a functional configuration of the second ultrasound diagnostic apparatus in an operating state.

FIG. 11 is a block diagram showing a functional configuration of the second ultrasound diagnostic apparatus in a charging state during operation.

FIG. 12 is a block diagram showing a functional configuration of the third ultrasound diagnostic apparatus in a standby state.

FIG. 13 is a diagram showing a schematic configuration of a second switch.

FIG. 14 is a block diagram showing a functional configuration of a conventional ultrasound diagnostic apparatus in a standby state.

### DETAILED DESCRIPTION OF EMBODIMENTS

Hereinafter, one or more embodiments of the radiation image capturing apparatus according to the present invention will be described with reference to the drawings. However, the scope of the invention is not limited to the disclosed embodiments.

The first to third embodiments according to the present invention are described in detail in order with reference to the attached drawings.

### First Embodiment

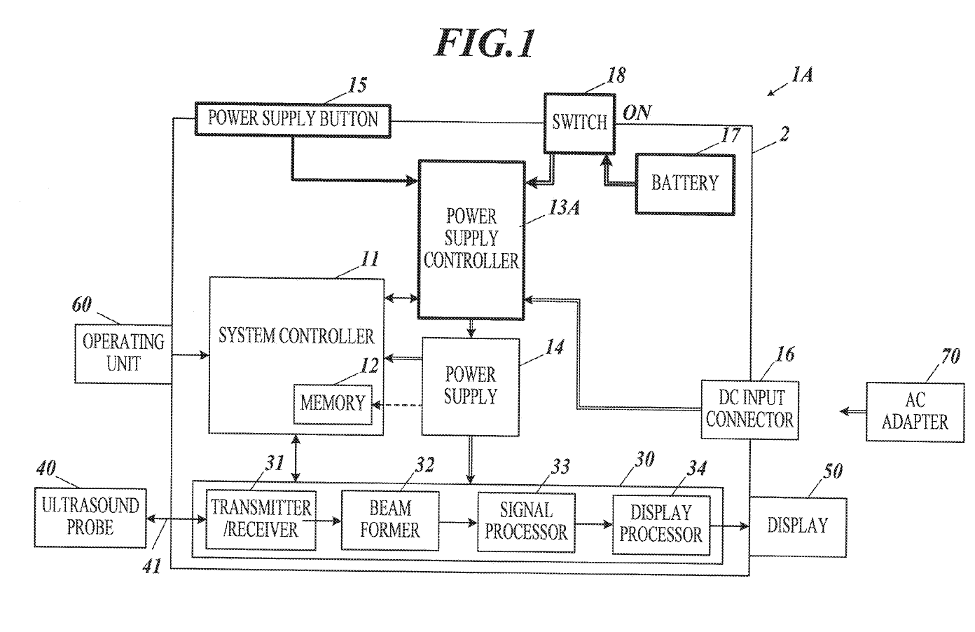

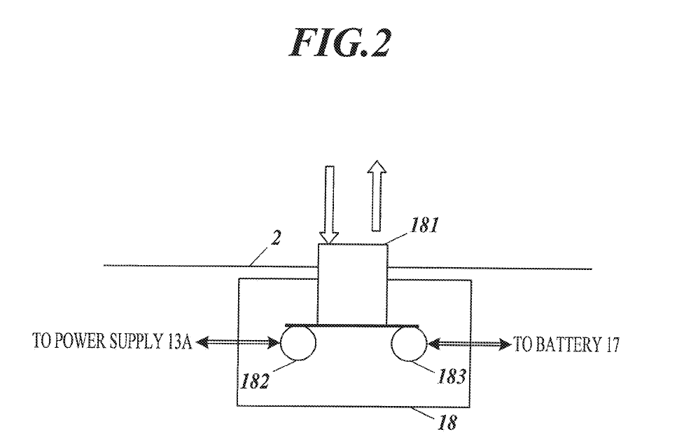

The first embodiment according to the present invention is described with reference to FIG. 1 to FIG. 4. First, the configuration of the apparatus according to the present embodiment is described with reference to FIG. 1 and FIG. 2. FIG. 1 is a block diagram showing a functional configuration of an ultrasound diagnostic apparatus 1A in a normal shutdown state according to the present embodiment. FIG. 2 is a diagram showing a schematic configuration of a switch 18.

The ultrasound diagnostic apparatus 1A as the medical image diagnostic apparatus of the present embodiment is used in medical facilities such as hospitals which have a commercial power supply, and outdoors where there is no commercial power supply. The ultrasound diagnostic apparatus 1A is a diagnostic apparatus which is portable by the user (examiner (physician, medical technician). As shown in FIG. 1, the ultrasound diagnostic apparatus 1A includes a housing 2, an ultrasound probe 40, a display 50, an operating unit 60, and an AC adapter 70.

The ultrasound probe 40 transmits ultrasound (transmitting ultrasound) to the subject such as a live body of a patient (not shown) and receives receiving ultrasound including reflecting ultrasound reflected from the subject and scattering ultrasound. The housing 2 is connected to the ultrasound probe 40 through the cable 41, and the electric driving signal is transmitted to the ultrasound probe 40 so that the ultrasound probe 40 transmits the transmitting ultrasound to the subject. In response to the receiving ultrasound from the subject received by the ultrasound probe 40, the housing 2 images an ultrasound image showing the internal state of the subject based on the electric receiving signal generated in the ultrasound probe 40.

A plurality of transducers (not shown) of the ultrasound probe 40 are arranged in a one dimensional array in an orienting direction. For example, according to the present embodiment, the ultrasound probe 40 provided with 192 transducers is used. The transducers may be arranged in a two-dimensional array. The number of transducers can be set freely. An electronic scanning method, or a mechanical scanning method, and a linear scanning method, a sector scanning method, or a convex scanning method can be employed in the ultrasound probe 40.

As shown in FIG. 1, the housing 2 includes a system controller 11, a power supply controller 13A, a power supply 14, a power supply button 15, a DC input connector 16 as an external power supply input unit, a battery 17 as a battery unit, and an ultrasound signal processor 30. The system controller 11 includes the volatile memory 12. The ultrasound signal processor 30 includes a transmitter/receiver 31, a beam forming unit 32, a signal processor 33, and a display processor 34.

For example, the operating unit 60 includes various switches, buttons, a track ball, a mouse, and a keyboard to input various commands such as instructions to start examination of the subject, measuring conditions, and data such as the subject information. The operating information according to the operating input is output to the system controller 11. Specifically, the operating unit 60 receives from the user input of executing ending and the selection information (shutdown or standby) of how to end when the ultrasound diagnostic apparatus 1A ends.

The transmitter/receiver 31 functions as the transmitter and performs the following. The transmitter/receiver 31 generates the driving signal as the electric signal corresponding to the driving voltage according to the driving voltage (driving voltage value) from the system controller 11, supplies the driving signal to the ultrasound probe 40 through the cable 41, and generates the transmitting ultrasound. The transmitter/receiver 31 as the transmitter is provided with a clock generating circuit, a delay circuit, and a pulse generating circuit, for example. The clock generating circuit is a circuit which generates a clock signal which determines transmitting timing and the transmitting frequency of the driving signal. The delay circuit is a circuit which sets the delay time for each individual path corresponding to each transducer, delays the transmitting timing of the driving signal in the amount of the set delay time, and focuses the transmitting beam composed by the transmitting ultrasound. The pulse generating circuit is a circuit which generates the pulse signal as the driving signal at a predetermined frequency. The transmitter/receiver 31 as described above drives a continuous portion (for example, 64) of a plurality of transducers (for example, 192) in the ultrasound probe 40 and generates the transmitting ultrasound. Then, the transmitter/receiver 31 scans the transducers driven each time the transmitting ultrasound is generated shifting in the orientation direction. With this, the ultrasound probe 40 transmits and receives the ultrasound according to the driving signal from the transmitter/receiver 31.

The transmitter/receiver 31 functions as the receiver which receives the electric receiving signal from the ultrasound probe 40 through the cable 41 according to control of the system controller 11. The transmitter/receiver 31 outputs the receiving signal of the transducers of the ultrasound probe 40.

The beam former 32 generates sound ray data by strengthening the receiving signal of the transducers input from the transmitter/receiver 31 according to the control of the system controller 11. For example, the beam former 32 includes an amplifier, an A/D (Analog to Digital) converting circuit, and a phase adding circuit. The amplifier is a circuit which amplifies the receiving signal at a preset amplifying percentage for each individual path corresponding to each transducer. The A/D converting circuit is a circuit which performs A/D conversion on the amplified receiving signal. The phase adding circuit is a circuit which provides a delay time for each individual path corresponding to each transducer to the A/D converted receiving signal to arrange the time phase, and adds the above (phase adding) to generate the sound ray data.

According to the control of the system controller 11, the signal processor 33 performs an envelope detecting process and a logarithmic amplification on the sound ray data input from the beam former 32, adjusts the dynamic range and gain to convert the brightness, and generates the B (Brightness) mode image data. That is, the B mode image data shows the strength of the receiving signal with the brightness. The signal processor 33 is able to generate the ultrasound image data of other diagnosis modes other than the B mode image data with the B mode as the diagnosis mode, for example, A (Amplitude) mode, M (motion) mode, pulse Doppler method, and color Doppler method.

The signal processor 33 includes an image memory (not shown) including a semiconductor memory such as a DRAM (Dynamic Random Access Memory). Under the control of the system controller 11, the signal processor 33 stores the generated B mode image data in the image memory in a unit of frames, and outputs the above as the image data of each frame.

Under the control of the system controller 11, the display processor 34 performs coordinate conversion on the frame image data input from the signal processor 33 to convert the above to image signals, and outputs the image signals to the display 50.

Various display apparatuses such as an LCD (Liquid Crystal Display), a CRT (Cathode-Ray Tube) display, an organic EL (Electronic Luminescence) display, an inorganic EL display and a plasma display may be employed as the display 50. Under the control of the system controller 11 through the ultrasound signal processor 30, the display 50 displays ultrasound images and the like on the display screen according to the image signals input from the display processor 34. Specifically, the display 50 displays the display screen information which receives from the user input to end the apparatus and input regarding the selection (shutdown or standby) of the method to end when the ultrasound diagnostic apparatus 1A ends.

For example, the system controller 11 includes the CPU (Central Processing Unit), the ROM (Read Only Memory), and the memory 12. The system controller 11 reads various process programs such as a system program stored in the ROM, deploys the program in the memory 12, and controls the units of the ultrasound diagnostic apparatus 1A according to the deployed program. The ROM includes a nonvolatile memory such as a semiconductor, and stores a system program in coordination with the ultrasound diagnostic apparatus 1A, various process programs which can be executed on the system program, and various data such as a gamma table. Such programs are stored in a computer readable program code format, and the CPU operates according to the program code. The memory 12 is a volatile storage such as a RAM (Random Access Memory), and forms a work area which temporarily stores various programs executed by the CPU and the data regarding such programs.

The power supply controller 13A is supplied with source power from the battery 17 or the AC adapter 70 (commercial power supply). The power supply controller 13A supplies the source power to the power supply 14, stands by for pressing of the power supply button 15, notifies the information of the power supply button 15 being pressed to the system controller 11, ends the ultrasound diagnostic apparatus 1A under the control of the system controller 11 (standby, shutdown), and starts the ultrasound diagnostic apparatus 1A. The power supply controller 13A may have various configurations depending on the complexity of control, and may be implemented by a digital circuit, a small CPU such as a microcomputer, or an IC dedicated to control of the power supply.

The power supply 14 is a power supply which supplies the source power to the system controller 11 and the ultrasound signal processor 30 (ultrasound probe 40, display 50, operating unit 60). Specifically, the power supply 14 supplies the source power (standby power supply) to the memory 12 when standing by, according to the instruction of the power supply controller 13A. The power supply 14 may include a regulator (DC-DC converter, series regulator). The regulator may be a circuit including a discreet component, or may be an IC or circuit module.

The power supply button 15 is provided on the surface of the housing 2. The power supply button 15 receives from the user pressing operation input as operation of turning the power on and off, and outputs the operation signal to the power supply controller 13A. The contents of operation received by the power supply button 15A includes starting after ending (standby, shutdown) the ultrasound diagnostic apparatus 1A, and ending (shutdown) while the ultrasound diagnostic apparatus 1A is started.

Here, the AC adapter 70 is electrically connected to the commercial power supply, converts the input commercial power supply from the alternating current to the direct current, and supplies the converted direct current as the source power to the DC input connector 16 of the connected device.

The DC input connector 16 is provided on the surface of the housing 2 and is an input connector to electrically connect the AC adapter 70. The DC input connector 16 outputs the direct current source power input from the electrically connected AC adaptor 70 to the power supply controller 13A.

The battery 17 is a secondary battery such as a lithium-ion battery internally mounted in the housing 2, and is able to charge source power from the AC adapter 70 and to output (discharge) source power to the power supply controller 13A. The battery 17 is electrically connected to the switch 18.

The switch 18 is provided on the surface of the housing 2, and is a switch which electrically connects and cuts the path between the battery 17 and the power supply controller 13A according to on/off operation by the user. The switch 18 is a push lock switch of two terminals shown in FIG. 2. The switch 18 shown in FIG. 2 includes an operating unit 181. When the operating unit 181 is pressed by the user, a contact 182 electrically connected to the power supply controller 13A is electrically connected to a contact 183 electrically connected to the battery 17. The contacts are connected locked to each other and the operating unit 181 is set to an on state. When the operating unit 181 in an on state is pressed by the user again, the lock is released, the electric connection between the contact 182 and the contact 183 is cut and the operating unit 181 is set to an off state. The switch 18 is not limited to a push lock switch, and may be a seesaw switch, a toggle switch or any other switch which electrically connects and cuts two contacts.

Some or all of the functions of the function block in each unit provided in the ultrasound diagnostic apparatus 1A can be implemented as a hardware circuit such as an integrated circuit. For example, the integrated circuit includes LSI (Large Scale Integration) and depending on the difference in degree of integration, the LSI may be called an IC (integrated Circuit), a system LSI, a super LSI, or an ultra LSI. The method of establishing an integrated circuit is not limited to an LSI, and can be implemented by a dedicated circuit or a general processor, or a reconfigurable processor in which the connection and setting of the circuit cell in the LSI or FPGA (Field Programmable Gate Array) can be reconfigured. Some or all of the functions in each functional block may be realized by software. In this case, the software may be stored in one or more among a storage medium such as a ROM, an optical disk or a hard disk, and the software is executed by a calculation processor.

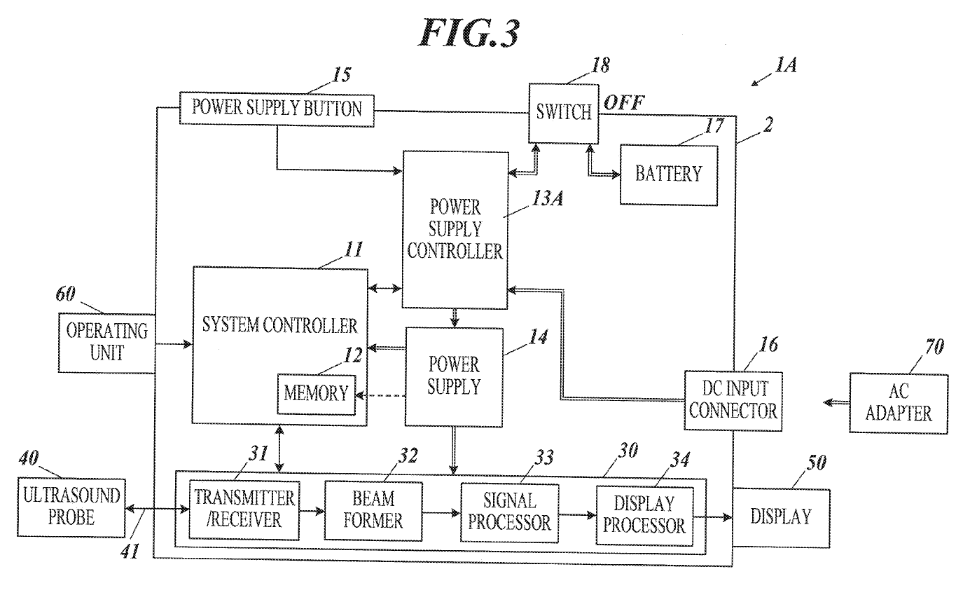

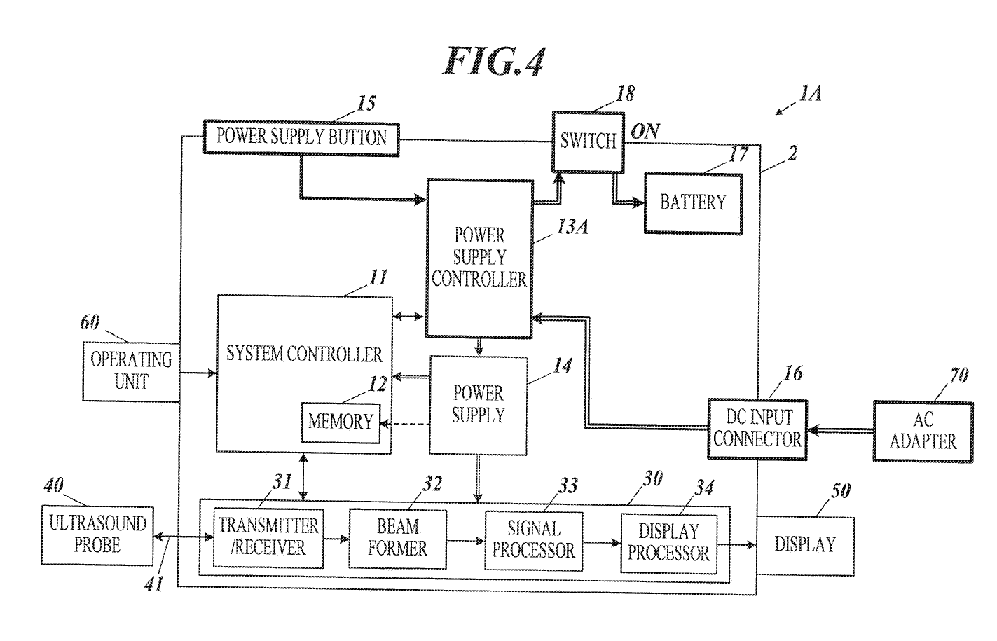

Next, the operation regarding the power supply of the ultrasound diagnostic apparatus 1A is described with reference to FIG. 1, FIG. 3, and FIG. 4. FIG. 3 is a block diagram showing a functional configuration of an ultrasound diagnostic apparatus 1A in a complete shutdown state. FIG. 4 is a block diagram showing a functional configuration in the ultrasound diagnostic apparatus 1A in a charged state during normal shutdown.

The ultrasound diagnostic apparatus 1A is already in operation and the switch 18 is turned on. The battery 17 is already charged.

When the system controller 11 of the ultrasound diagnostic apparatus 1A in the operating state receives input of an end instruction of the ultrasound diagnostic apparatus 1A from the user through the operating unit 60, the display screen information to select the method to end the ultrasound diagnostic apparatus 1A is displayed on the display 50. Then, the system controller 11 receives input of selection of shutdown or standby as the method to end the ultrasound diagnostic apparatus 1A from the user through the operating unit 60.

For example, in order to stop operation of the ultrasound diagnostic apparatus 1A for a long amount of time, when the shutdown is selected or the power supply button 15 is turned off, the system controller 11 controls the power supply controller 13A to end the ultrasound diagnostic apparatus 1A in a normal shutdown state, as shown in FIG. 1. In the ultrasound diagnostic apparatus 1A in the normal shutdown state, the source power output from the battery 17 is supplied to the power supply controller 13A through the switch 18 in the on state as shown with the bold line showing charging or operating. As shown with the bold line, the power supply controller 13A is in a standby state which is able to receive the starting operation from the power supply button 15 using the source power being supplied. Therefore, the consumed power of the ultrasound diagnostic apparatus 1A in the normal shutdown state is lower than when started but is mainly consumed by the power supply controller 13A.

When the user turns off the switch 18 in the ultrasound diagnostic apparatus 1A in the normal shutdown state, as shown in FIG. 3, the ultrasound diagnostic apparatus 1A advances to the complete shutdown state.

In the ultrasound diagnostic apparatus 1A in the complete shutdown state, since the switch 18 is electrically cut, there is no bold line, and the source power is not supplied from the battery 17 to the power supply controller 13A. Therefore, the power consumed by the ultrasound diagnostic apparatus 1A in the complete shutdown state is much lower than the power consumed by the ultrasound diagnostic apparatus 1A in the normal shutdown state. However, in the complete shutdown state, even if the power supply button 15 is pressed, the ultrasound diagnostic apparatus 1A does not start.

When the AC adapter 70 connected to the commercial power supply is electrically connected to the DC input connector 16 in the ultrasound diagnostic apparatus 1A in the normal shutdown state, as shown in FIG. 4, the ultrasound diagnostic apparatus 1A advances to the charged state during normal shutdown. The source power output from the AC adapter 70 is supplied and charged to the battery 17 through the DC input connector 16, power supply controller 13A, and switch 18 in the on state in this order. When the switch 18 is turned off and the ultrasound diagnostic apparatus 1A advances to the complete shutdown state in the ultrasound diagnostic apparatus 1A in the normal shutdown state, even if the AC adapter 70 connected to the commercial power supply is electrically connected to the DC input connector 16, the battery 17 is not charged.

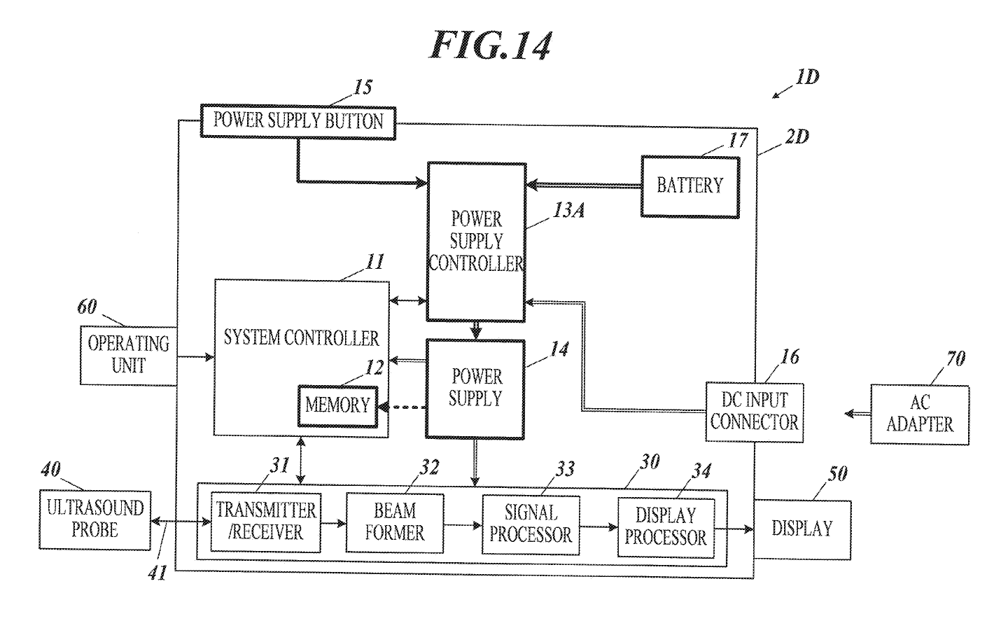

When the display screen information to select the end method while the ultrasound diagnostic apparatus 1A is in operation is displayed on the display 50 and standby is selected in order to temporarily pause the operation and then operate the ultrasound diagnostic apparatus 1A after a short period of time passes, the system controller 11 sets the power supply controller 13A to a standby state similar to the ultrasound diagnostic apparatus 1D shown in FIG. 14. The power supply controller 13A maintains the standby state which can receive the operation of the power supply button 15 with the source power supplied from the battery 17 through the switch 18 turned on, supplies the source power to the memory 12 through the power supply 14, and maintains the stored state of the data stored in the memory 12. In the ultrasound diagnostic apparatus 1A in the standby state, when the switch 18 is turned off, the data stored in the memory 12 is erased, and thus the user does not turn off the switch 18.

According to the present embodiment, the ultrasound diagnostic apparatus 1A includes a power supply 14 which supplies source power to each unit of the ultrasound diagnostic apparatus 1A, a power supply button 15 which receives operation to end or start the ultrasound diagnostic apparatus 1A, a power supply controller 13A which controls the reception of input to the power supply button 15 to supply power to the power supply 14 and to control the power supply 14, a battery 17 which is internally mounted in the ultrasound diagnostic apparatus 1A and which supplies source power to the power supply controller 13A, and a switch 18 which receives operation of on or off and which electrically connects or cuts the path between the battery 17 and the power supply controller 13A according to the operation.

Therefore, when it is assumed that the ultrasound diagnostic apparatus 1A in the normal shutdown state will not be used for a long amount of time without detaching the battery 17 from the ultrasound diagnostic apparatus 1A or the battery 17 cannot be charged from the external power supply, the switch 18 is turned off. With this, the ultrasound diagnostic apparatus 1A is completely shut down, and the consumed power after ending the ultrasound diagnostic apparatus 1A can be easily reduced. After travelling for a long amount of time, the diagnosis can be performed for a length of time that is originally possible with the charged amount in the battery 17. Since there is no need to detach the battery 17 from the ultrasound diagnostic apparatus 1A, the ultrasound diagnostic apparatus 1A can be transported with the battery 17, and it is possible to prevent leaving only the battery 17.

The ultrasound diagnostic apparatus 1A includes a DC input connector 16 which supplies source power input from the AC adapter 70 outside the ultrasound diagnostic apparatus 1A to the power supply controller 13A. The power supply controller 13A supplies the source power input from the DC input connector 16 to the battery 17 through the switch 18. Therefore, even in the complete shutdown state, the switch 18 is turned on (normal shutdown state) and the battery 17 can be surely charged.

The ultrasound diagnostic apparatus 1A includes an operating unit 60 which receives input to end the ultrasound diagnostic apparatus 1A by shutdown or standby, and a system controller 11 which performs an end process of the ultrasound diagnostic apparatus 1A by shutdown or standby according to the operation input on the operating unit 60 or the power supply button 15. Therefore, the ultrasound diagnostic apparatus 1A ends in the desired state between shutdown or standby in response to operation of the operating unit 60 or the power supply button 15.

The ultrasound diagnostic apparatus 1A includes a volatile memory 12 which stores data. Standby is a state in which source power is supplied to the power supply controller 13A, the power supply 14, and the memory 12, and the data stored in the memory 12 is held. Therefore, by ending in a standby state, the data stored in the memory 12 can be held, and after restart, the ultrasound diagnostic apparatus 1A can be readily restored using the above data.

The normal shutdown is a state in which source power is supplied to the power supply controller 13A. Therefore, in the normal shutdown, the controller stands by for start operation of the power supply button 15, and when the start operation is performed, the control to supply power to the power supply 14 is immediately performed.

### Second Embodiment

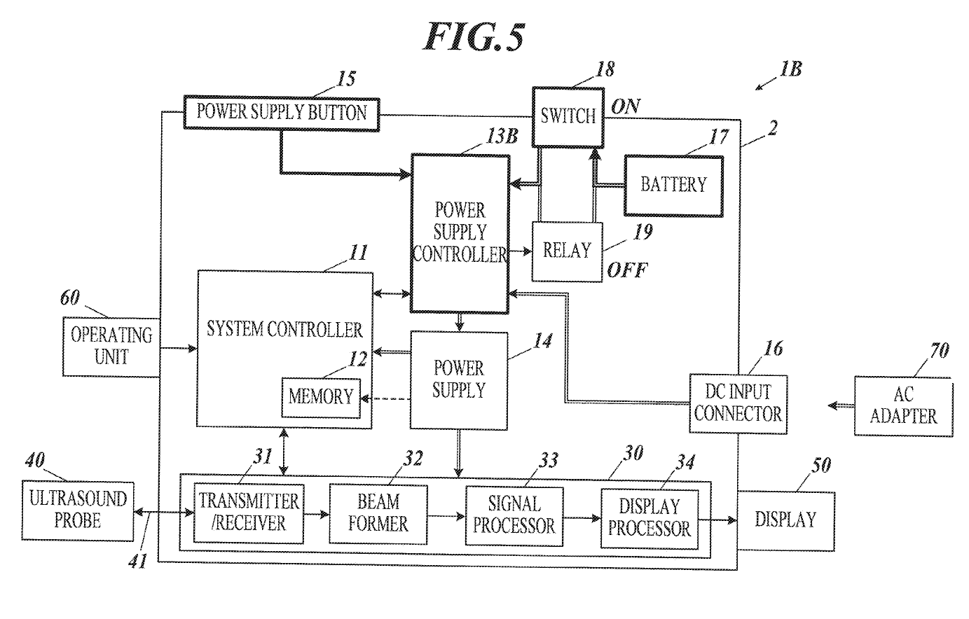

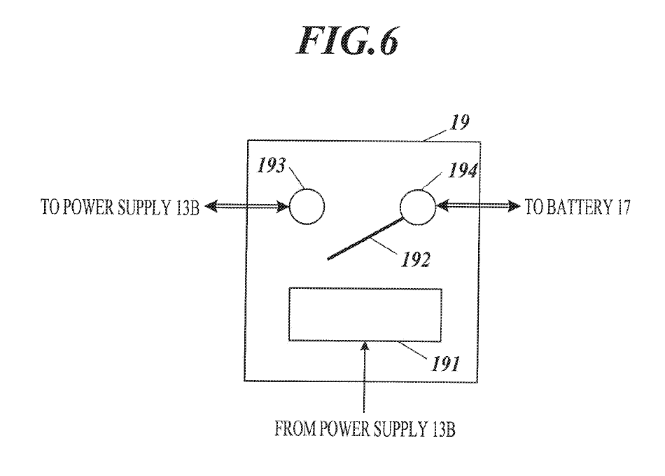

The second embodiment of the present invention is described with reference to FIG. 5 to FIG. 11. First, the configuration of the apparatus according to the present embodiment is described with reference to FIG. 5 and FIG. 6. FIG. 5 is a block diagram showing a functional configuration of the ultrasound diagnostic apparatus 1B in the normal shutdown state. FIG. 6 is a diagram showing a schematic configuration of the relay 19.

As shown in FIG. 5, the ultrasound diagnostic apparatus 1B according to the present embodiment includes a power supply controller 13B instead of the power supply controller 13A of the ultrasound diagnostic apparatus 1A according to the first embodiment, and an additional component which is a relay 19. Therefore, the components in the ultrasound diagnostic apparatus 1B similar to those in the ultrasound diagnostic apparatus 1A are provided with the same reference numerals and the description is omitted. The different components are mainly described.

The power supply controller 13B is supplied with source power from a battery 17 or an AC adapter 70 (commercial power supply). The power supply controller 13B performs processes such as supplying source power to the power supply 14, stands by for the pressing of the power supply button 15, ends (standby, shutdown) and starts the ultrasound diagnostic apparatus 1A according to the system controller 11 or the pressing of the power supply button 15 and controls on and off of the relay 19.

The relay 19 is provided parallel to the switch 18 between the battery 17 and the power supply controller 13B. According to the control from the power supply controller 13B, the relay 19 switches the electric connection or discharge of the path between the battery 17 and the power supply controller 13B. The relay 19 is a single stipple relay, for example. When the electric current or the electric voltage is applied to the control terminal of the relay 19 by the power supply controller 13B, the path between the battery 17 and the power supply controller 13B is electrically connected and turned on. When the electric current or the electric voltage is not applied to the control terminal of the relay 19 by the power supply controller 13B, the path between the battery 17 and the power supply controller 13B is electrically cut and turned off.

As shown in FIG. 6, the relay 19 includes an operating unit 191 and a contact 192. When there is an electric current or an electric voltage from the power supply controller 13B, the operating unit 191 turns on the contact 192, and electrically connects the contact 193 electrically connected to the power supply controller 13B to the contact 194 electrically connected to the battery 17. When there is no electric current or electric voltage from the power supply controller 13B, the operating unit 191 turns off the contact 192 and cuts the electric connection between the contact 193 and the contact 194.

The relay used in the relay 19 is largely classified between a mechanical type and a semiconductor type. For example, in the mechanical type relay 19, the operating unit 191 is to be an excitation coil and magnet, and the contact 192 is to be the mechanical switch turned on and off according to the movement of the magnet of the operating unit 191. For example, in the semiconductor type relay 19, the operating unit 191 is to be the light emitting portion such as the LED (Light Emitting Diode) and the contact 192 is to be the electric switch such as the Photo MOS (Metal-Oxide Semiconductor) switch turned on and off according to whether the light is emitted in the operating unit 191.

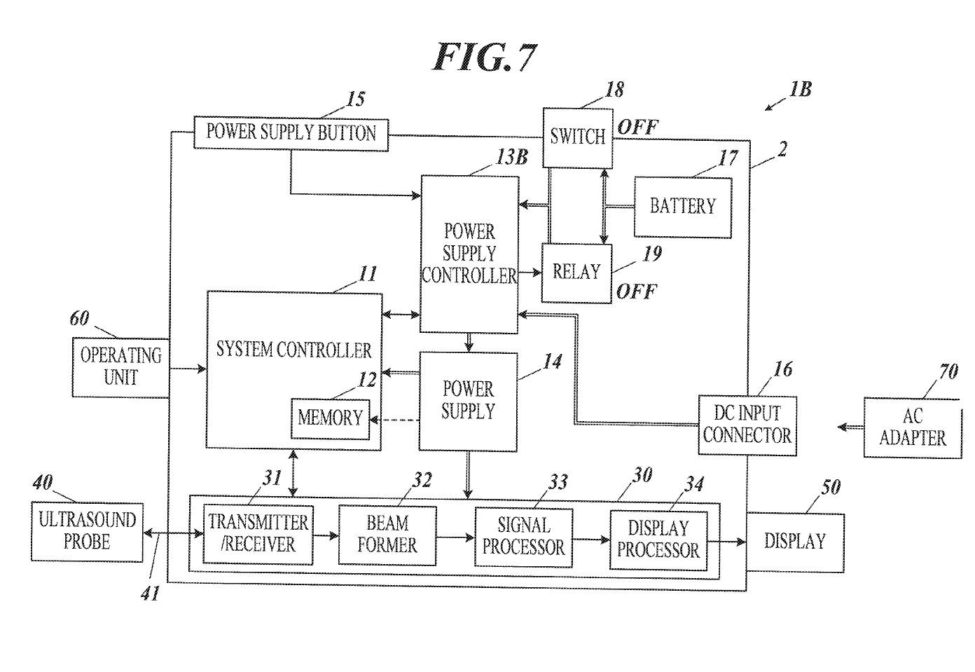

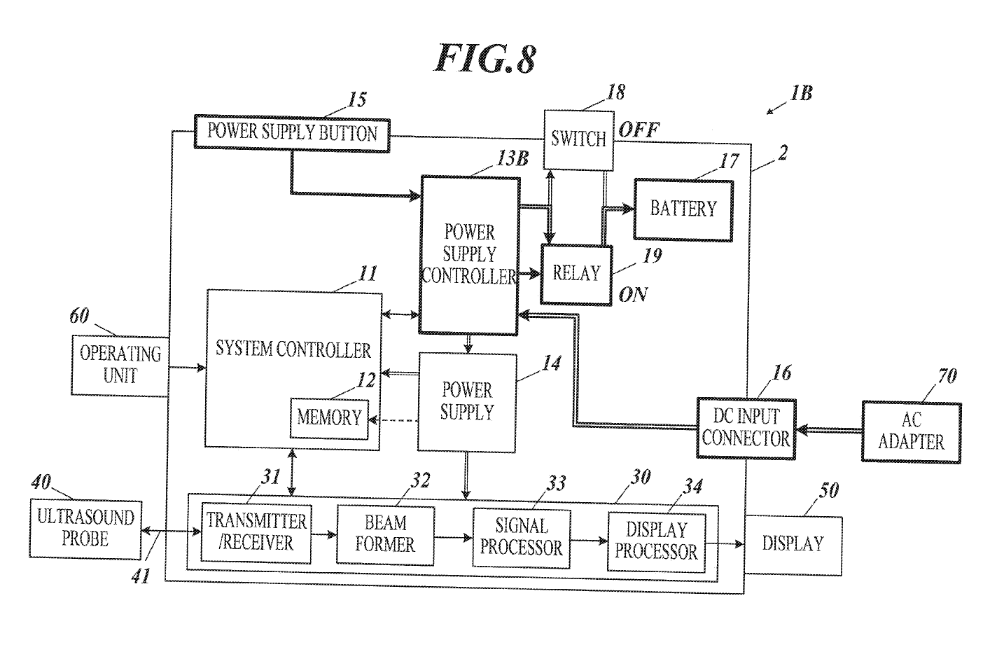

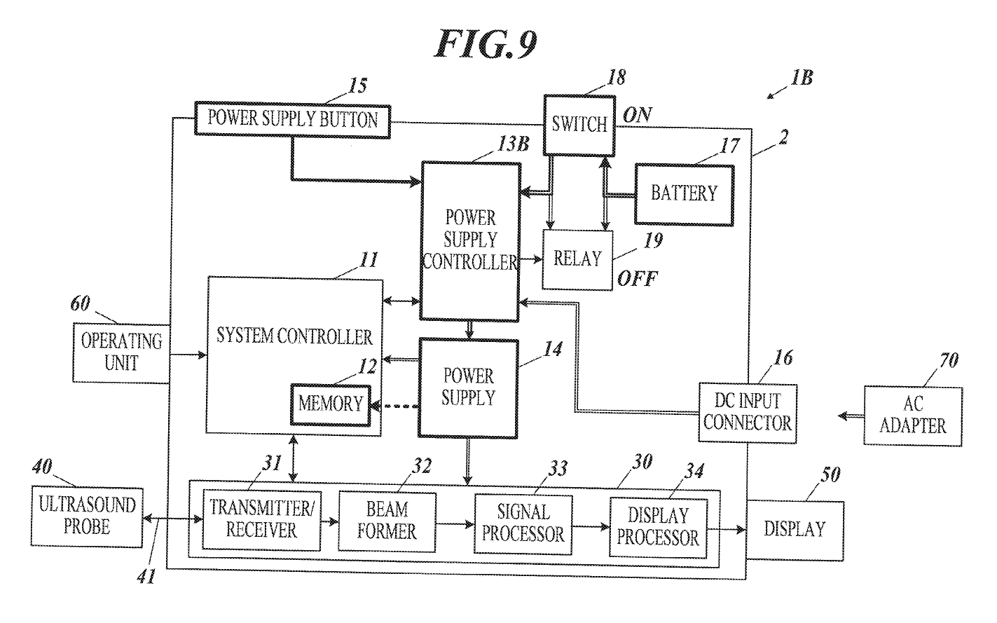

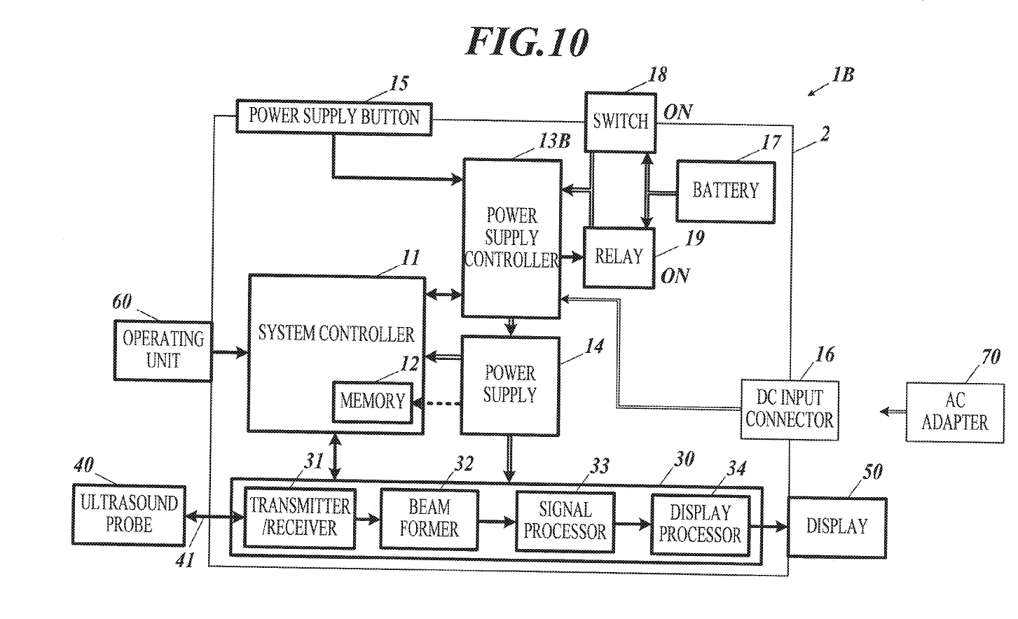

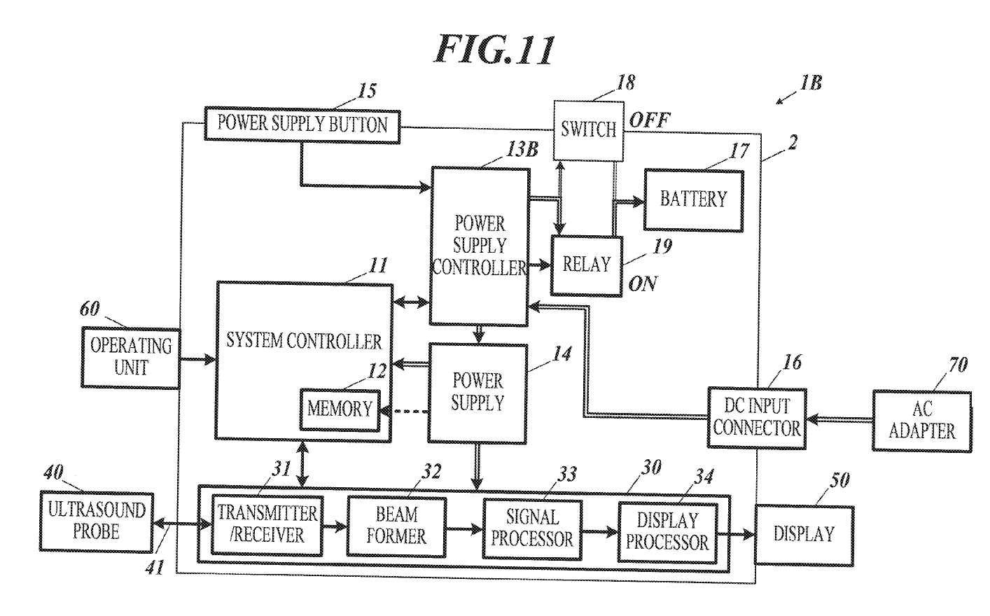

Next, the operation regarding the power supply of the ultrasound diagnostic apparatus 1B is described with reference to FIG. 5, and FIG. 7 to FIG. 11. FIG. 7 is a block diagram showing a functional configuration of the ultrasound diagnostic apparatus 1B in a complete shutdown state. FIG. 8 is a block diagram showing a functional configuration of the ultrasound diagnostic apparatus 1B in a charged state during normal shutdown. FIG. 9 is a block diagram showing a functional configuration of the ultrasound diagnostic apparatus 1B in the standby state. FIG. 10 is a block diagram showing the functional configuration of the ultrasound diagnostic apparatus 1B in an operating state. FIG. 11 is a block diagram showing the functional configuration of the ultrasound diagnostic apparatus 1B in a charged state during operation.

According to FIG. 5, the ultrasound diagnostic apparatus 1B is in operation and the switch 18 is turned on. The battery 17 is charged.

When the input of the end instruction of the ultrasound diagnostic apparatus 1B is received from the user through the operating unit 60 in the ultrasound diagnostic apparatus 1B in operation, the system controller 11 displays the display screen information for selection of the end method of the ultrasound diagnostic apparatus 1B on the display 50. The system controller 11 receives input of selection between shutdown or standby as the end method of the ultrasound diagnostic apparatus 1B from the user through the operating unit 60.

When the shutdown is selected or the power supply button 15 is turned off, as shown in FIG. 5, the system controller 11 controls the power supply controller 13B to end the ultrasound diagnostic apparatus 1B in a normal shutdown state. In the ultrasound diagnostic apparatus 1B in the normal shutdown state, the source power output from the battery 17 is supplied to the power supply controller 13B through the switch 18 in the on state as shown with the bold line showing charging or operating. As shown with the bold line, the power supply controller 13B uses the supplied source power to maintain the standby state which is able to receive the starting operation from the power supply button 15 and turns off the relay 19. Therefore, the power consumed by the ultrasound diagnostic apparatus 1B in the normal shutdown state is lower than the amount consumed in starting, but the power is mainly consumed by the power supply controller 13B.

When the user turns off the switch 18 in the normal shutdown state of the ultrasound diagnostic apparatus 1B, as shown in FIG. 7, the ultrasound diagnostic apparatus 1B advances to the complete shutdown state.

In the ultrasound diagnostic apparatus 1B in the complete shutdown state, since the switch 18 is electrically cut, there is no bold line and the source power is not supplied from the battery 17 to the power supply controller 13B. Therefore, the power consumed by the ultrasound diagnostic apparatus 1B in the complete shutdown state is further decreased than the power consumed by the ultrasound diagnostic apparatus 1B in the normal shutdown state. In the complete shutdown state, even if the power supply button 15 is pressed, the ultrasound diagnostic apparatus 1B is not started.

When the AC adapter 70 connected to the commercial power supply is electrically connected to the DC input connector 16 in the ultrasound diagnostic apparatus 1B in the complete shutdown state, as shown in FIG. 8, the ultrasound diagnostic apparatus 1B advances to the charged state in the normal shutdown. The source power output from the AC adapter 70 is supplied to the power supply controller 13B through the DC input connector 16 as shown with the bold line. As shown with the bold line, the power supply controller 13B uses the supplied source power to maintain the standby state which can receive the starting operation from the power supply button 15 and turns on the relay 19 again. Therefore, the source power output from the power supply controller 13B is supplied to the battery 17 through the relay 19 and charged. When the AC adapter 70 connected to the commercial power supply is electrically connected to the DC input connector 16 in the ultrasound diagnostic apparatus 1B in the normal shutdown state, similarly, the source power output from the AC adapter 70 is supplied and charged to the battery 17 through the DC input connector 16, the power supply controller 13B, and the relay 19 in the on state, in this order.

When the display screen information for the selection of the method to end while the ultrasound diagnostic apparatus 1B is operating is displayed on the display 50 and the standby is selected and input, the system controller 11 controls the power supply controller 13B to be in a standby state as shown in FIG. 9. The power supply controller 13B uses the source power supplied from the battery 17 through the switch 18 in the on state so that the power supply button 15 is set to an operable standby state, the source power is supplied to the memory 12 through the power supply 14, and the data stored in the memory 12 is held. The power supply controller 13B may control the relay 19 to be on or off, but preferably sets to off to save power. If the switch 18 is turned off when the relay 19 is off in the ultrasound diagnostic apparatus 1B in the standby state, the data stored in the memory 12 is erased. If the switch 18 is turned off even when the relay 19 is on in the ultrasound diagnostic apparatus 1B in the standby state, the data stored in the memory 12 is not erased.

In order to start the ultrasound diagnostic apparatus 1B in the ultrasound diagnostic apparatus 1B in the complete shutdown state, the switch 18 is turned on and then the ultrasound diagnostic apparatus 1B advances to the normal shutdown state. When the power supply button 15 is turned on after the ultrasound diagnostic apparatus 1B advances to the normal shutdown state, as shown in FIG. 10, the source power output from the battery 17 is supplied to the power supply controller 13B through the switch 18 turned on. The power supply controller 13B supplies the source power to the power supply 14. The power supply 14 supplies power to each unit of the ultrasound diagnostic apparatus 1B such as the system controller 11, the ultrasound signal processor 30, the display 50, and the operating unit 60. The power supply controller 13B turns on the relay 19. The system controller 11 starts and operates the ultrasound diagnostic apparatus 1B. When the ultrasound diagnostic apparatus 1B is operated, regardless of whether the switch 18 is on or off, the source power output from the battery 17 is continuously supplied to the power supply 14 through the relay 19 turned on and the power supply controller 13B.

Other ways to start the ultrasound diagnostic apparatus 1B in the complete shutdown state include electrically connecting the AC adapter 70 connected to the commercial power supply to the DC input connector 16. When the AC adapter 70 connected to the commercial power supply is electrically connected to the DC input connector 16 in the ultrasound diagnostic apparatus 1B in the complete shutdown state, similar to FIG. 8, the source power output from the AC adapter 70 is supplied and charged in the battery 17 through the DC input connector 16, the power supply controller 13B, and the relay 19 turned on, and the ultrasound diagnostic apparatus 1B advances to the charged state in normal shutdown. Then, when the power supply button 15 is turned on, as shown in FIG. 11, the source power output from the AC adapter 70 is supplied to the power supply controller 13B through the DC input connector 16. The power supply controller 13B supplies source power to the power supply 14. The power supply 14 supplies power to each unit of the ultrasound diagnostic apparatus 1B such as the system controller 11, the ultrasound signal processor 30, the display 50, and the operating unit 60, and turns on the relay 19. The system controller 11 starts and operates the ultrasound diagnostic apparatus 1B. When the ultrasound diagnostic apparatus 1B is starting, regardless of whether the switch 18 is on or off, the source power output from the AC adapter 70 continues to be supplied to the battery 17 through the DC input connector 16, the power supply controller 13B, and the relay 19 turned on.

According to the above-described embodiment, the ultrasound diagnostic apparatus 1B includes a relay 19 which is provided parallel with the switch 18 between the battery 17 and the power supply controller 13B and which connects or cuts the path between the battery 17 and the power supply controller 13B in response to control of on or off from the power supply controller 13B. When the switch 18 is on and the source power is input from the DC input connector 16, the power supply controller 13B turns on the relay 19 and supplies the input source power to the battery 17 through the relay 19. Therefore, even when the ultrasound diagnostic apparatus 1B is in a complete shutdown state, the ultrasound diagnostic apparatus 1B can advance to a charged state in normal shutdown with the switch 18 turned off, and the battery 17 can be surely charged.

When the ultrasound diagnostic apparatus 1B is in operation, the power supply controller 13B turns on the relay 19. Therefore, even when the switch 18 is pressed while the ultrasound diagnostic apparatus 1B is in operation, it is possible to prevent the ultrasound diagnostic apparatus 1B from advancing to the complete shutdown state.

When the ultrasound diagnostic apparatus 1B ends during standby, the power supply controller 13B turns off the relay 19. Therefore, the consumed power can be reduced in the ultrasound diagnostic apparatus 1B in the standby state.

### Third Embodiment

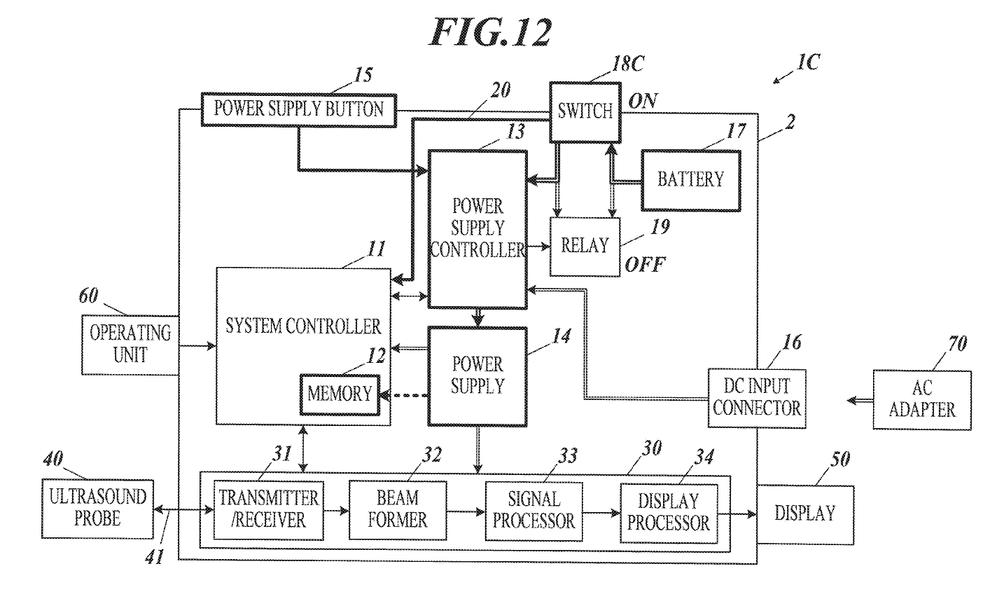

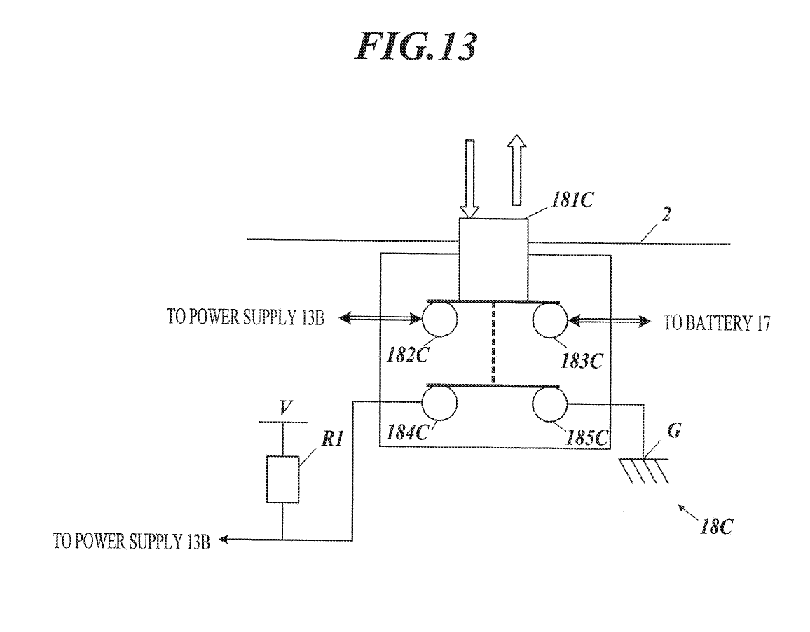

The third embodiment according to the present invention is described with reference to FIG. 12 and FIG. 13. First, the apparatus configuration of the present embodiment is described with reference to FIG. 12 and FIG. 13. FIG. 12 is a block diagram showing a functional configuration of the ultrasound diagnostic apparatus 1C in a standby state. FIG. 13 is a diagram showing a schematic configuration of the switch 18C.

As shown in FIG. 12, the ultrasound diagnostic apparatus 1C according to the present embodiment includes a switch 18C as a switch and a notifying unit instead of the switch 18 in the ultrasound diagnostic apparatus 1B according to the second embodiment. Moreover, a path 20 is added to the ultrasound diagnostic apparatus 1C according to the present embodiment. The same reference numerals are applied to the components of the ultrasound diagnostic apparatus 1C which are the same as those of the ultrasound diagnostic apparatus 1B, and the description is omitted. The different portions are mainly described.

The switch 18C is provided on the surface of the housing 2. The user turns the switch 18 on and off to electrically connect or cut the path between the battery 17 and the power supply controller 13B and the signal showing the on or off state is output to the power supply controller 13B. The path 20 is an electric path provided between the switch 18C and the power supply controller 13B.

The switch 18C is to be a push lock switch with four terminals as shown in FIG. 13. The switch 18C shown in FIG. 13C includes an operating unit 181C. When the operating unit 181C is pressed by the user, the operating unit 181 electrically connects a contact 182C electrically connected to the power supply controller 13B to a contact 183C electrically connected to the battery 17, and in coordination with this electric connection, electrically connects a contact 184C connected to a power supply voltage V through a pullup resistance R1 with a contact 185C connected to a ground G, and turns on the switch in a locked state with the connection continuing. With the switch 18C turned on, a low-level voltage signal from a ground G is output to the path 20 (power supply controller 13B) through the contact 185C, operating unit 181C, and the contact 184C. The low-level voltage signal is a signal showing that the switch 18C is turned on.

When the operating unit 181C in the on state is pressed again by the user, the electric connection between the contact 182C and the contact 183C is cut, and in coordination with the above, the electric connection between the contact 184C and the contact 185C is cut. With this, the switch is turned off. When the switch 18C is turned off, a high-level voltage signal is output to the path 20 (power supply controller 13B) from the power supply voltage V through the pullup resistance R1. The high-level voltage signal is a signal showing that the switch 18C is turned off. The switch 18C is not limited to a push lock switch, and may be a seesaw switch, a toggle switch, or any other switch which electrically connects or cuts two pairs of contacts.

Next, the operation regarding the power supply of the ultrasound diagnostic apparatus 1C is described with reference to FIG. 12.

According to the ultrasound diagnostic apparatus 1C, the user does not select how to end the ultrasound diagnostic apparatus 1C through the operating unit 60. The ultrasound diagnostic apparatus 1C automatically selects how to end the ultrasound diagnostic apparatus 1C according to whether the switch 18C is on or off.

The ultrasound diagnostic apparatus 1C is in operation and the switch 18 is turned on. The battery 17 is fully charged.

When the user inputs end through the operating unit 60 or the power supply button 15 is turned off in the ultrasound diagnostic apparatus 1C in operation, the system controller 11 ends the ultrasound diagnostic apparatus 1C in a complete shutdown state or a standby state according to the signal input from the switch 18C through the path 20.

For example, the user may turn off the switch 18C when the ultrasound diagnostic apparatus 1C is in operation as in the ultrasound diagnostic apparatus 1B shown in FIG. 10. Here, since the relay 19 is turned on, the supply of the source power output from the battery 17 to the power supply 14 through the relay 19 and the power supply controller 13B continues, and the operating state of the ultrasound diagnostic apparatus 1C continues. The signal input from the switch 18C to the power supply controller 13B through the path 20 is a signal which shows the switch 18C is off. When the user inputs end through the operating unit 60 or the power supply button 15 is turned off in the ultrasound diagnostic apparatus 1C in the operating state, the system controller 11 controls the power supply controller 13B to turn off the relay 19 according to the input signal showing the switch 18C is off, and the ultrasound diagnostic apparatus 1C ends in the complete shutdown state. The source power from the battery 17 is not provided because the switch 18C and the relay 19 are turned off.

For example, the switch 18C is turned on in the ultrasound diagnostic apparatus 1C in the operating state similar to the ultrasound diagnostic apparatus 1B shown in FIG. 10. Here, since the relay 19 is turned on, the supply of the source power output from the battery 17 to the power supply 14 through the relay 19 and the power supply controller 13B continues, and the ultrasound diagnostic apparatus 1C continues operation. The signal input from the switch 18C to the power supply controller 13B through the path 20 is the signal showing the switch 18C is turned on. When the user inputs end through the operating unit 60 or the power supply button 15 is turned off in the ultrasound diagnostic apparatus 1C in operation, as shown in FIG. 12, the system controller 11 controls the power supply controller 13B to turn the relay 19 on or off according to the input signal showing the switch 18C is off, and ends the ultrasound diagnostic apparatus 1C in the standby state. The source power output from the battery 17 is supplied to the power supply controller 13B through the switch 18C turned on, and the power is supplied to the memory 12 through the power supply 14. Here, the power supply controller 13B may turn on the relay 19, but preferably, the relay 19 is turned off to save power.

Similar to the ultrasound diagnostic apparatus 1B in the standby state as shown in FIG. 8, the AC adapter 70 electrically connected to the commercial power supply can be electrically connected to the DC input connector 16 so that the ultrasound diagnostic apparatus 1C is in the charged state during standby.

According to the above-described embodiment, the ultrasound diagnostic apparatus 1C includes the operating unit 60 which receives operation to end the ultrasound diagnostic apparatus 1C and the system controller 11 which performs the end process of the ultrasound diagnostic apparatus 1C according to the operation input by the operating unit 60 or the power supply button 15. When the switch 18C is on, it is notified to the system controller 11 that the switch 18C is on. The system controller 11 performs the end process of the ultrasound diagnostic apparatus 1C to set to standby in response to the operation to end input by the operating unit 60 or the power supply button 15 when there is a notification that the switch 18C is on. Therefore, even if the user does not select standby as the method to end the apparatus, the apparatus ends as standby automatically in response to the on state of the switch 18C and the burden of the user decreases.

When the switch 18C is off, it is notified to the system controller 11 that the switch 18C is off. The system controller 11 performs the end process of the ultrasound diagnostic apparatus 1C to set to shutdown in response to the operation to end input by the operation unit 60 or the power supply button 15 when there is a notification that the switch 18C is off. Therefore, even if the user does not select shutdown as the method to end the apparatus, the apparatus ends as shutdown automatically in response to the off state of the switch 18C and the burden of the user decreases.

The embodiments shown above are merely examples of preferred embodiments of the ultrasound diagnostic apparatus according to the present invention, and the present invention is not limited to the above.

For example, according to the above-described embodiment, the switch 18, the switch 18C, and the relay 19 are applied to the ultrasound diagnostic apparatus including the battery 17, but the present invention is not limited to the above. For example, the configuration of the above-described embodiments such as the switch 18, the switch 18C, and the relay 19 can be applied to other medical image diagnostic apparatuses which include a battery and perform processes on medical images such as an image diagnostic apparatus which performs processes such as generate and display radiation images, or an image diagnostic apparatus which performs processes such as generate and display electrocardiograms.

The detailed configuration and operation composing the ultrasound diagnostic apparatus 1A, 1B, and 1C according to the present embodiment can be suitably changed without leaving the scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2017-092724 filed on May 9, 2017 is incorporated herein by reference in its entirety.

What is claimed is:

1. A medical image diagnostic apparatus which processes a medical image of a subject, the apparatus comprising:
a power supply which supplies power to each unit of the medical image diagnostic apparatus;
a power supply button which receives operation to end or start the medical image diagnostic apparatus;
a power supply controller which stands by to receive input of the power supply button and which supplies power to the power supply and controls the power supply;
a battery which is mounted in the medical image diagnostic apparatus and which supplies power to the power supply controller; and
a switch which receives operation to switch between on and off, and which electrically connects or cuts a path between the battery and the power supply controller in response to the operation.

2. The medical image diagnostic apparatus according to claim 1, further comprising an external power supply input unit which supplies source power input from devices outside the medical image diagnostic apparatus to the power supply controller,
wherein, the power supply controller supplies the source power input by the external power supply input unit to the battery through the switch.

3. The medical image diagnostic apparatus according to claim 2, further comprising a relay which is provided between the battery and the power source controller and parallel to the switch, and which connects or cuts the path between the battery and the power supply controller in response to control by the power supply controller switching between on and off,
wherein, the power supply controller turns on the relay and supplies the input source power to the battery through the relay when the switch is off and the source power is input by the external power supply input unit.

4. The medical image diagnostic apparatus according to claim 3, wherein the power supply controller turns on the relay when the medical image diagnostic apparatus is in operation.

5. The medical image diagnostic apparatus according to claim 3, wherein the power supply controller turns off the relay when the medical image diagnostic apparatus ends in a standby state.

6. The medical image diagnostic apparatus according to claim 1, further comprising,
an operating unit which receives input to end the medical image diagnostic apparatus in a shutdown state or a standby state; and
a system controller which ends the medical image diagnostic apparatus in a shutdown state or a standby state according to an operation input by the operating unit or the power supply button.

7. The medical image diagnostic apparatus according to claim 1, further comprising,
an operating unit which receives input to end the medical image diagnostic apparatus;
a system controller which performs an end process of the medical image diagnostic apparatus in response to operation input by the operating unit or the power supply button; and
a notification unit which notifies to the system controller that the switch is on when the switch is on,
wherein the system controller ends the medical diagnostic apparatus in a standby state in response to operation to end which is input by the operating unit or the power supply button when there is a notification that the switch is on.

8. The medical image diagnostic apparatus according to claim 7, wherein the notification unit notifies that the switch is off to the system controller when the switch is off, and
the system controller ends the medical image diagnostic apparatus in a shutdown state in response to an operation to end which is input by the operating unit or the power supply button when there is a notification that the switch is off.

9. The medical image diagnostic apparatus according to claim 5, further comprising a volatile storage which stores data,
wherein power is supplied to the power supply controller, the power supply, and the storage in the standby state and the data stored in the storage is maintained.

10. The medical image diagnostic apparatus according to claim 6, wherein power is supplied to the power supply controller in the shutdown state.
